(19) United States
(12) Patent Application Publication
(10) Pub. No.: US 2021/0330584 A1
(43) Pub. Date: Oct. 28, 2021

(54) TOPICAL PREPARATIONS OF DRUG DELIVERY FOR NASAL AND SINUS IRRIGATION

(71) Applicant: Topical Sinus Therapeutics, Inc., Camarillo, CA (US)

(72) Inventors: Russell N. Reitz, Camarillo, CA (US); John C. Tarrant, Camarillo, CA (US)

(73) Assignee: Topical Sinus Therapeutics, Inc., Camarillo, CA (US)

(21) Appl. No.: 17/238,037

(22) Filed: Apr. 22, 2021

Related U.S. Application Data
(60) Provisional application No. 63/014,053, filed on Apr. 22, 2020.

(51) Int. Cl. A61K 9/00; A61K 47/36; A61K 45/06; A61K 9/46; A61M 11/00

(52) U.S. Cl. CPC A61K 9/0014; A61K 47/36; A61M 11/007; A61K 9/0043; A61K 9/0007; A61K 45/06

(57) ABSTRACT

Embodiments of the application are directed toward a liquid topical sinus therapy for a patient, comprising a medicated formulation including at least one medication, and a device for delivering the medicated formulation to the patient's nostrils, nasal passages, or sinuses. The medicated formulation may include drugs selected from the group consisting of corticosteroids, antibiotics, antifungals, antihistamines as well as herbal and alternative medications. Dosage forms include powders, tablets and gels which facilitate clinical testing and enable patient compliance.

### RELATED APPLICATIONS

This application claims priority to U.S. Provisional Patent Application No. 63/014,053 filed on Apr. 22, 2020, the content of which is incorporated herein by reference in its entirety.

### TECHNICAL FIELD

The present disclosure relates generally to targeted drug administration, and more specifically, some embodiments relate to the targeted topical administration of nasal and sinus irrigation.

### BACKGROUND

When a patient visits their doctor, presenting symptoms of sinusitis, their doctor is likely to prescribe a 7-10-day oral antibiotic regimen such as clarithromycin, augmentin or sulfamethoxazole/trimethoprim possibly combined with a nasal spray such as Nasonex or Flonase. However, if symptoms persist, a physician may prescribe a nasal/sinus saline irrigation to flush out allergens, mucus, and foreign microbes. This patient is most often instructed to flush the solution into each nostril twice daily. In some more severe cases, physicians will prescribe medication to be added to the saline irrigation. Drugs that may be included are corticosteroids to reduce inflammation or an anti-infective to topically treat a sinus infection. To those familiar with the art of irrigation, no standardized dose exists in the treatment of these patients. Furthermore, when drug mixed with saline is irrigated into the nasal pharynx, most of the medication is expelled through the other nostril and is wasted down the drain. Many patients find it cumbersome and inconvenient to irrigate their sinuses with medication due to the fact that several steps are involved to complete that process. The patient must be in the privacy of a restroom with a sink in order to administrate the therapy. And this therapy is usually required to be administered twice daily for at least 30 days. In the following paragraphs technology will be disclosed which will improve patient compliance and create a process by which standardized dosing may occur.

Chronic Rhino Sinusitis (CRS) is an exceedingly difficult disease to treat. Some physicians view it as an infectious disease, while others view CRS as an inflammatory disease. Regardless of clinical opinion, CRS is difficult to treat because of the structure of the human anatomy. Within the cranium, sinuses are hollow cavities which receive truly little blood flow. In order for oral medications to be effective, they must be absorbed into the bloodstream and then travel to the mucus membranes surrounding the sinuses to reduce inflammation or treat an infection.

Oral treatments and intravenous therapies are beneficial when treating acute cases of sinusitis; however, they are of little benefit in severe cases of chronic sinusitis. In addition, bacterial CRS infections are particularly resistant to medications due to the bacterial formation of biofilm. Bacterial colonies create biofilm, a defense mechanism, made up of various bivalent cations such as iron, calcium, zinc, and other minerals set within an extracellular matrix composed of polymeric substances, which make it particularly difficult for medications to penetrate the bacterial colony. In addition, gram (−) negative bacteria are particularly more likely to be resistant to medical treatment.

When administering oral or IV drugs to treat the inflamed and infected tissues of CRS patients, adequate drug concentrations might not be achieved within the hollow sinus cavities. By the time, an oral or IV drug reaches the infected tissue, the drug concentration may not be sufficient enough to transfer out of the blood system of the sinus tissue and transfer into the hollow sinus cavities and have a meaningful impact on inflammation, treat the infection or penetrate biofilm. In addition, there are many other side effects associated with high dose treatments of oral and intravenous therapies. This is why so many physicians resort to topical therapies when treating Chronic Rhino Sinusitis and other nasal related diseases. Topical sinus therapy implies that medication is applied directly to the site of infection or inflammation so that the patient may obtain relief.

Postoperative lavage of the paranasal sinus is a recognized adjuvant in the treatment of chronic rhinosinusitis, which reduces morbidity and improves local healing. In addition allowing the association of topical medications that may be carried to the paranasal sinuses along with saline increases the penetration of the drug into the difficult to reach cavernous matrix. Many physicians recommend that sinus patients rinse their nasal passages with saline to flush our excess mucus and allergens residing in the nose and nasal pharynx. Studies indicate that for patients who have had sinus surgery, these irrigations may also enter the sinus cavities temporarily. Some physicians have even gone to the extent of prescribing compounded medications that are added to saline and then flushed through the nasal passages in hopes of landing the medication inside the sinus cavities.

ENT surgeons routinely prescribe off label use of various drugs (antibiotics, corticosteroids, antifungals, etc.) to be mixed with saline and flushed into the nasal passages of their sinus patients. These solutions are then compounded at various compounding pharmacies across the United States and throughout the world with no standardization as to the dosing or the administration of the drugs or therapy.

For example, in one solution physicians have prescribed Mupirocin. Mupirocin (brand name BACTROBAN®) ointment is indicated for the topical treatment of impetigo due to susceptible isolates of Staphylococcus aureus (S. aureus) and Streptococcus pyogenes (S. pyogenes). Mupirocin nasal ointment is used to treat or prevent infections in the nose due to certain strains of Staphylococcus aureus bacteria. This medicine works by killing bacteria or preventing their growth. This medication is currently available in a 22 gm tube of ointment. Each gram of medication contains 20 mg of mupirocin and the balance is polyethylene glycol. A typical off-label prescription may read “squeeze out about one inch of ointment into your irrigation bottle, add 8 ounces of saline and shake. After shaking, squirt the resulting suspension into your nostrils. Do this twice per day for 30 days.” When a patient performs this administration, the dosing result is less than accurate.

In another solution the physician may prescribe budesonide, a corticosteroid indicated for: Maintenance treatment of asthma as prophylactic therapy in adult and pediatric patients six years of age or older. Because this product is FDA approved it is widely available. However, even though budesonide is indicated for the treatment of asthma, physicians prescribe it off-label with the instructions to their patient to open the ampule of liquid medication and squeeze it into their rinse bottle, add 8 ounces of saline and mix. The resulting rinse mixture is adequate (0.5 mg of budesonide) however this dose has never been tested or FDA approved for nasal or sinus conditions. And budesonide may create unwanted side effects such as bruising easily, chills, colds, cough, hoarseness, fever, flu-like symptoms, sneezing, blurred vision, and sore throat. Ironically, some of these side effects are the very indications the doctor is hoping to treat with this therapy.

In yet another example, tobramycin (brand name TOBI®) is an aminoglycoside antibacterial indicated for the management of cystic fibrosis in adults and pediatric patients 6 years of age and older with pseudomonas aeruginosa infections. Tobramycin, inhalation solution, FDA-approved final product, non-compounded, unit dose form, is administered through DME, per 300 mg. These 5 ml vials may be prescribed off-label for sinus patients where the patient is instructed to open the ampule and squeeze the solution into their rinse device and add 8 ounces of saline, mix, and then irrigate each nostril. However, with a generic AWP cost ranging between $52 per dose to $158 per dose for branded ampoules, a BID (twice per day) administration regimen is cost prohibitive for most patients.

### BRIEF DESCRIPTION OF THE DRAWINGS

FIG. 1 illustrates a medication for topical sinus therapy comprising liquid in glass or plastic pharmaceutical grade ampules, in accordance with an embodiment of the application.

FIG. 2 illustrates a medication for topical sinus therapy in the form of a pharmaceutical grade sterile powder in packets or sachets (with or without salts), in accordance with another embodiment of the application.

FIGS. 3 and 4 illustrate a medication delivery system comprising a syringe, tube, vial, or a disposable cartridge of pharmaceutical grade water-soluble gel from a package containing a plurality of these items, in accordance with an embodiment of the application.

FIGS. 5 and 6 illustrate a further medication for topical sinus therapy in the form of a package of dissolvable effervescent tablets, in accordance with an embodiment of the application. The tablets may or may not be effervescent. The tablet will dissolve. It may also be a gelatin capsule where the capsule is opened and its contents are placed into water, or a dissolvable gel cap or a caplet or a liquid-gel capsule.

FIG. 7 illustrates a medication delivery system comprising a NeilMed® type rinse bottle, in accordance with an embodiment of the application.

FIG. 8 illustrates a medication delivery system comprising a Hydropulse®, in accordance with an embodiment of the application. FIG. 20 illustrates a medication delivery system comprising a SinuPulse® 500.

FIG. 9 illustrates a medication delivery system comprising a Neti Pot, in accordance with an embodiment of the application.

FIG. 10 illustrates a medication delivery system comprising a Navage®, in accordance with an embodiment of the application.

FIG. 11 illustrates a medication delivery system comprising a surgical syringe, in accordance with an embodiment of the application.

FIG. 12 illustrates a medication delivery system comprising another surgical syringe, in accordance with an embodiment of the application.

FIGS. 13-15 illustrate medication delivery systems in the form of different catheters, in accordance with further embodiments of the application. In one example the catheter is attached to a syringe.

FIG. 16 illustrates a medication delivery system comprising a squeeze bottle, in accordance with an embodiment of the application.

FIG. 17 illustrates another medication delivery system comprising a nosepiece attached to a flexible tube of such length so as to reach the bottom of a standard 500 ml purified water bottle. The nosepiece includes threading which allows the nose piece to attach to any standard water bottle. A tablet 230 is shown comprising a medicated formulation to be dissolved in the water. FIG. 19 illustrates a foil blister pack 400 containing a plurality of medicated formulation tablets 410.

FIG. 18 illustrates a further medication delivery system comprising a screw thread adapter for attachment between a water bottle and nosepiece.

FIG. 21 illustrates a MAD (Mucosal Atomization Device) 600 with a conical nose plug attached.

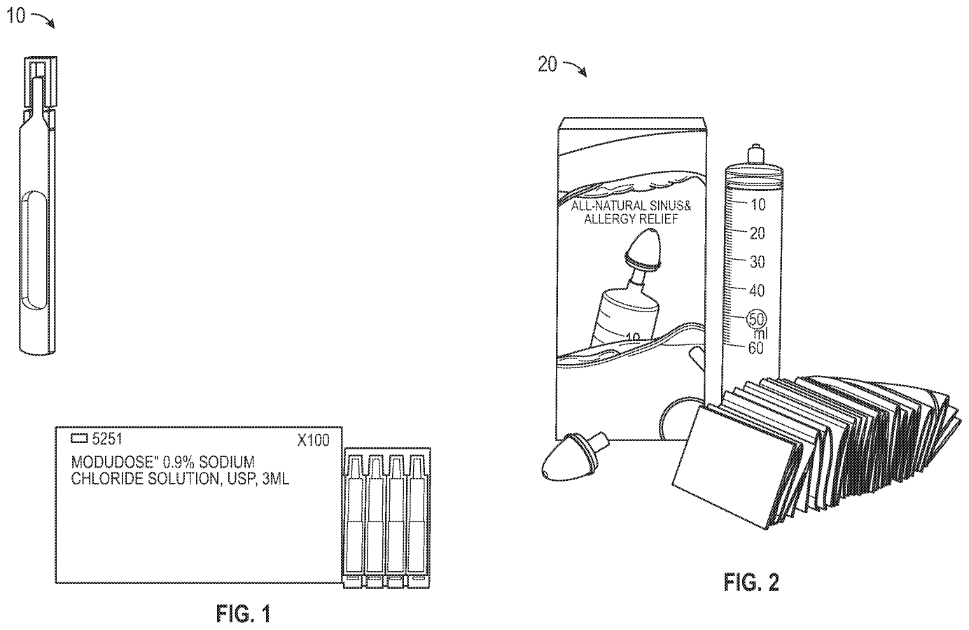

### DETAILED DESCRIPTION OF THE EMBODIMENTS

In view of the above, there exists a therapeutic need for standardization, scientific testing and FDA approval of safe and effective dosing of topical sinus irrigation therapy. There also exists a patient need for a cost effective, easily administered, FDA evaluated dose, which is approved for a nasal/sinus medicated irrigation drug delivery system to be prescribed by Ear, Nose and Throat physicians as well as other physicians treating sinus disease.

Embodiments in this application will provide for an easier administration of drug delivery resulting in improved patient compliance with this therapy.

In some embodiments of this invention the drug may be in tablet form. In other embodiments of this invention the drug may be in powder form. And in yet another embodiment of this invention the drug may be in gel formulation.

In all embodiments set forth herein, the medication(s) may or may not include NaCl. In embodiments where NaCl is included in the medication(s) formulation, the dose form (tablet, powder, or gel) is then added to water. In embodiments where NaCl is not included in the medication(s) formulation, the dose form (tablet, powder, or gel) is then added to premixed saline (NaCl).

Additional embodiments involve enhancements made to topical sinus therapy that allow medications to coat the nasal passages and sinuses better than when mixed with saline alone. Further improvements involve creating a formulation in such a way as to allow the medication to adhere to the infected and/or inflamed tissues longer than a normal medicated saline solution.

The nasal/sinus formulations provided in this detailed document include nasal irrigation fluids, powders, granules, pellets, sachets, gels, vials, syringes, tablets, and effervescent tablets comprising corticosteroids, antibiotics, antifungals, antihistamines, and alternative medicines. Methods of topical treatment included here treat acute sinusitis, chronic sinusitis, nasal/sinus polyps, allergic rhinitis and nasal congestion. Methods of delivery of the formulations and fluids to the sinuses, methods of coating the sinuses, and the treatment of chronic sinusitis, allergic rhinitis, nasal, and sinus polyps, as well as acute sinusitis are part of this invention.

Some embodiments of this invention involve standardizing the dosing of: (i) anti-inflammatory medication, including but not limited to mometasone furoate monohydrate, mometasone furoate, budesonide, micronized fluticasone propionate, micronized fluticasone furoate, etc.), (ii) antibiotic medications including but not limited to mupirocin, tobramycin, clarithromycin, levofloxacin, etc., and (iii) antifungal medications including but not limited to itraconazole, voriconazole, posaconazole, fluconazole, etc. and (iv) antihistamines including but not limited to fexofenadine, loratadine, diphenhydramine, azelastine, etc. and (v) alternative/herbal medications such as menthol, eucalyptus, manuka honey and methylglyoxal. Manuka Honey and its ingredients have an unusually high level of methylglyoxal (MGO) formed from dihydroxyacetone (DHA) which correlates with antibacterial activity.

FIG. 2 depicts another embodiment wherein the medication 20 for topical sinus therapy is in the form of a pharmaceutical grade powder in packets or sachets. During use, the patient mixes the dry medicated powder with water or saline to form a solution, which is delivered to the treatment area using a syringe having a plunger on one end and a nosepiece on the other end.

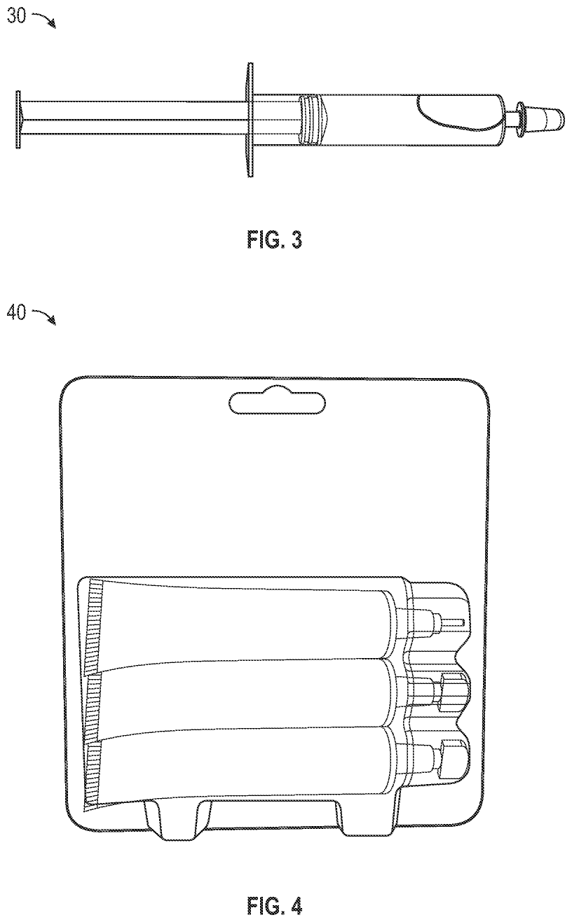

FIGS. 3 and 4 illustrate an embodiment of a medication delivery system 30 comprising a syringe, tube, or vial (FIG. 3) holding a disposable cartridge of pharmaceutical grade water-soluble gel from a package 40 containing a plurality of cartridges (FIG. 4). The water-soluble gel may be squeezed out into a bottle of water or saline and then mixed (swirled) to create a medicated saline irrigation.

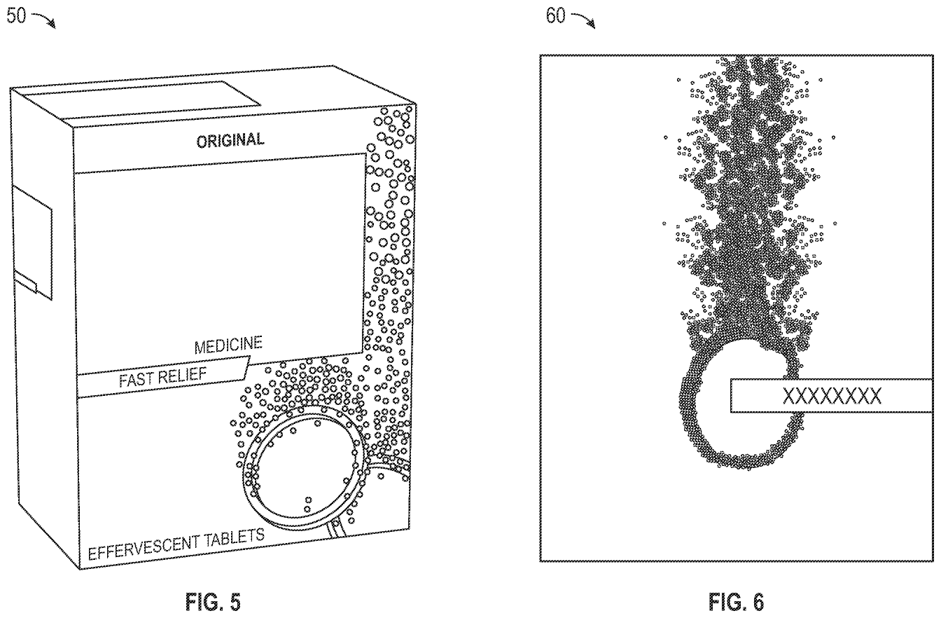

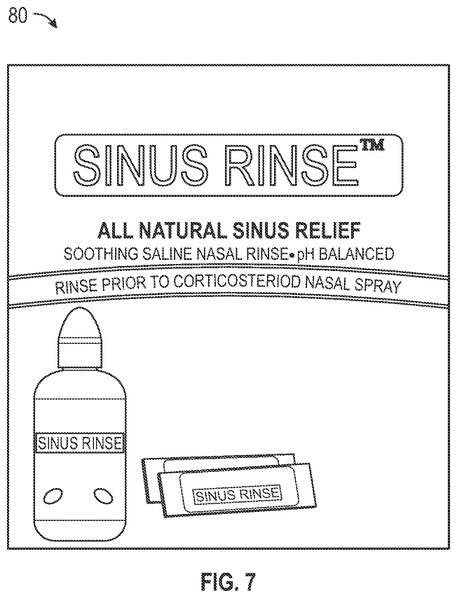

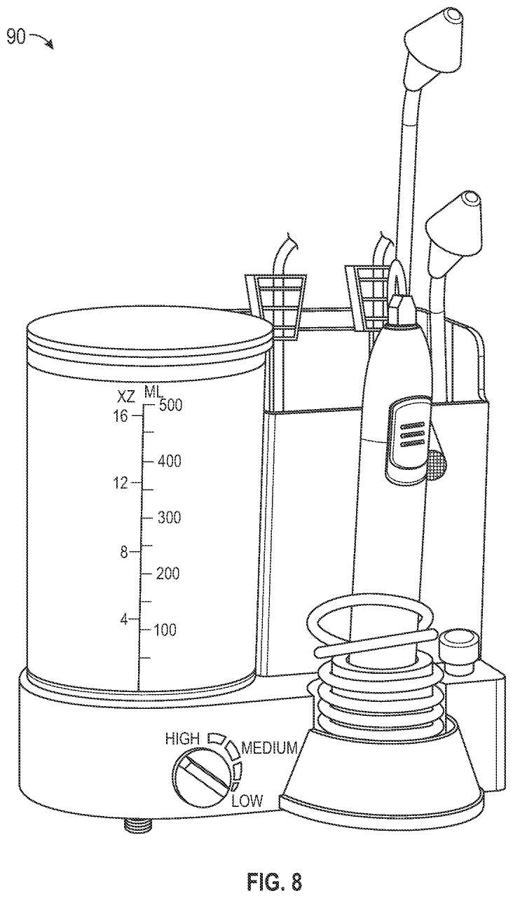

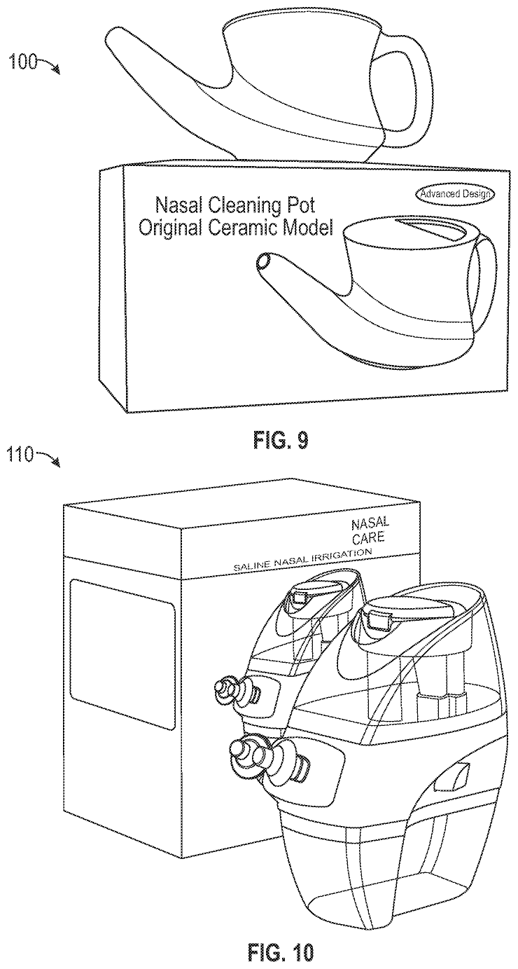

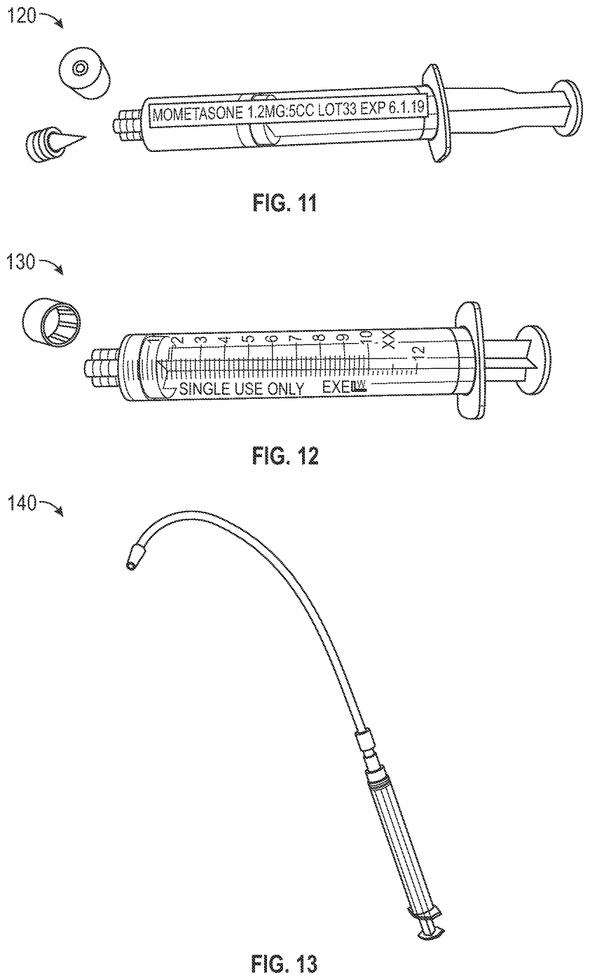

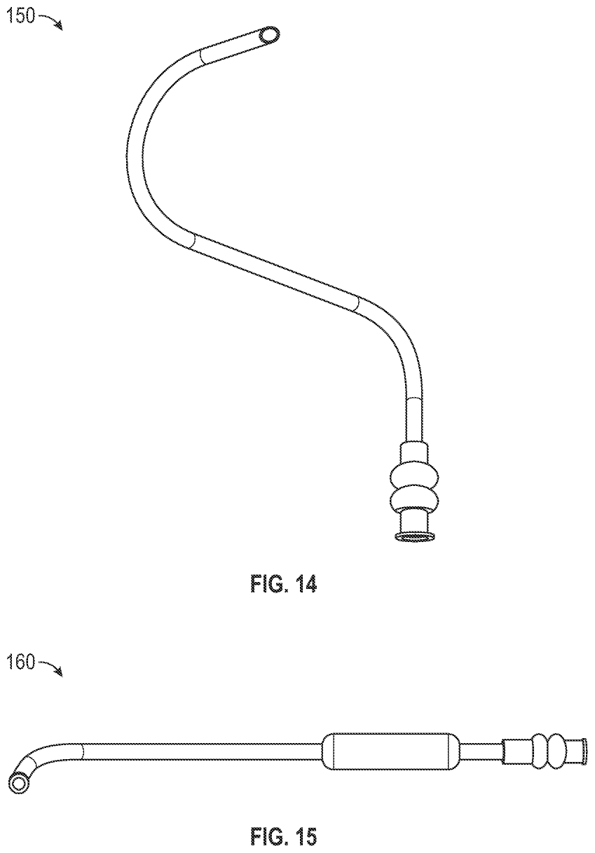

Referring to FIGS. 5 and 6, in further embodiments the medication for topical sinus therapy may come in the form of a package 50 of dissolvable effervescent tablets 60, which may be dropped into water or saline to form a medicated saline irrigation. In some cases, the tablet size may vary (i.e. vitamin shaped) such that it will fit through the opening of a standard water bottle or nasal rinse bottle. The medicated saline irrigation described with respect to FIGS. 3-6 can then applied directly to the nasal passages and/or sinuses via an irrigation device such as a syringe (FIG. 3), a NeilMed® type rinse bottle 80 (FIG. 7), a Hydropulse® 90 (FIG. 8), a Neti Pot 100 (FIG. 9), a Navage® 110 (FIG. 10), surgical syringe 120 (FIG. 11), surgical syringe 130 (FIG. 12), catheter 140 (FIG. 13), catheter 150 (FIG. 14), catheter 160 (FIG. 15), MAD (Mucosal Atomization Device), Sinupulse®, purified water bottle with a conical nose plug attachment or the like. Standardized doses of these medications with these devices may now be tested for FDA approval to be used by physicians and their patients for the treatment of inflammation, infections, allergies, polyps and other related sinus conditions.

### Medicated Irrigation Dosage Forms

As set forth above, the medication may be provided in a powder form packaged in a packet or sachet (FIG. 2), and then mixed with water, liquid saline or powdered saline and water. After mixing or swirling the solution/suspension, the patient may insert the medicated liquid into the nasal passages by following the directions of the irrigation device of her choice. Suitable irrigation systems include without limitation, a standard 500 ml purified water bottle with a conical nosepiece attachment, a syringe (FIG. 3), NeilMed® type rinse bottle 80 (FIG. 7), Hydropulse® 90 (FIG. 8), Neti Pot 100 (FIG. 9), Navage® 110 (FIG. 10), Sinupulse® or MAD (Mucosal Atomization Device). Any of the syringes described herein, such as those depicted in FIGS. 3, 11 and 12, may include a conical nose plug for one nostril to facilitate delivery of the medicated saline irrigation. The nosepiece may be made of silicone, rubber, latex, plastic, or other suitable material.

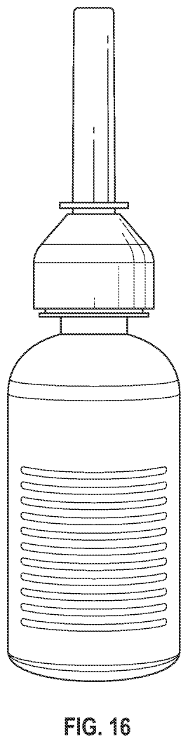

In other embodiments, as described herein and above, the medicated irrigation is provided in a water-soluble gel packaged in a tube or syringe or plastic vial, and then mixed with water, liquid saline or powdered saline and water. After mixing or swirling the solution/suspension, the patient may insert the medicated liquid into the nasal passages by following the directions of the irrigation device of her choice. Suitable irrigation systems are described herein with respect to FIGS. 3 and 7-20.

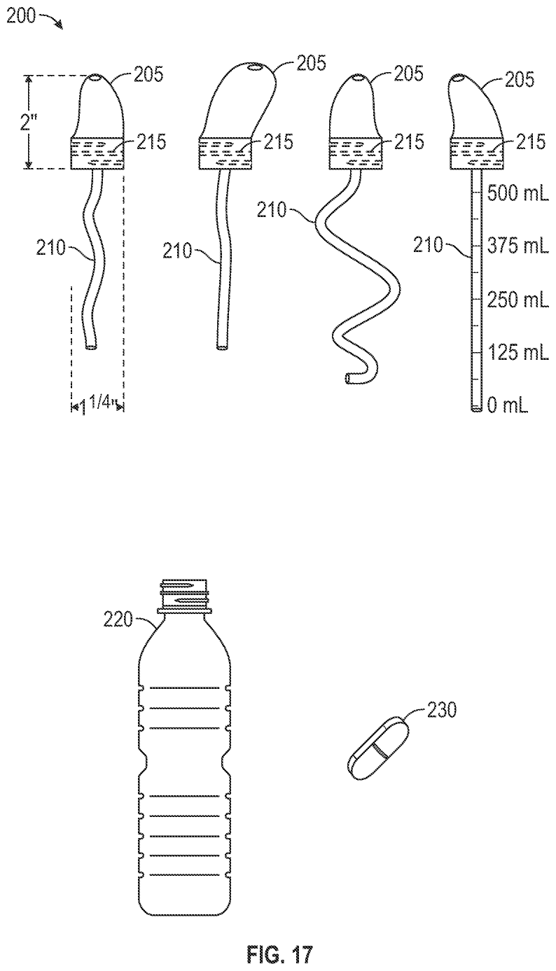

FIG. 17 illustrates another medication delivery system 200 comprising a conical nosepiece 205 attached to a flexible tube 210 of such length so as to reach the bottom of a standard 500 ml purified water bottle 220. The nosepiece includes internal threading 215 which allows the nose piece to attach to any standard purified water bottle via its standard external threading 225. The nose plug 205 may be straight or bent at an angle. In the illustrated embodiment, the drug is in the form of a tablet 230 to be dissolved by dropping it into the water bottle 220. In other embodiments, the drug may be in powder form or in a gel formulation. In some embodiments, the nosepiece 205 can have a conical tapered shape, and may include volume indication on the flexible tube 210 (as depicted in FIG. 17). In further embodiments, the nosepiece 205 may include an anti-siphon valve and tubing.

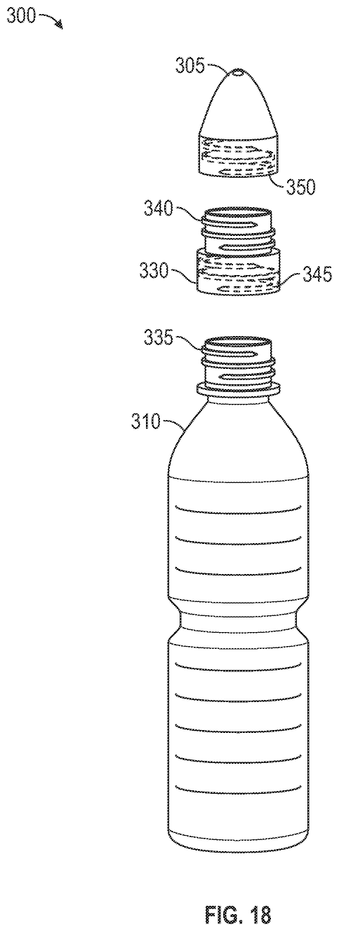

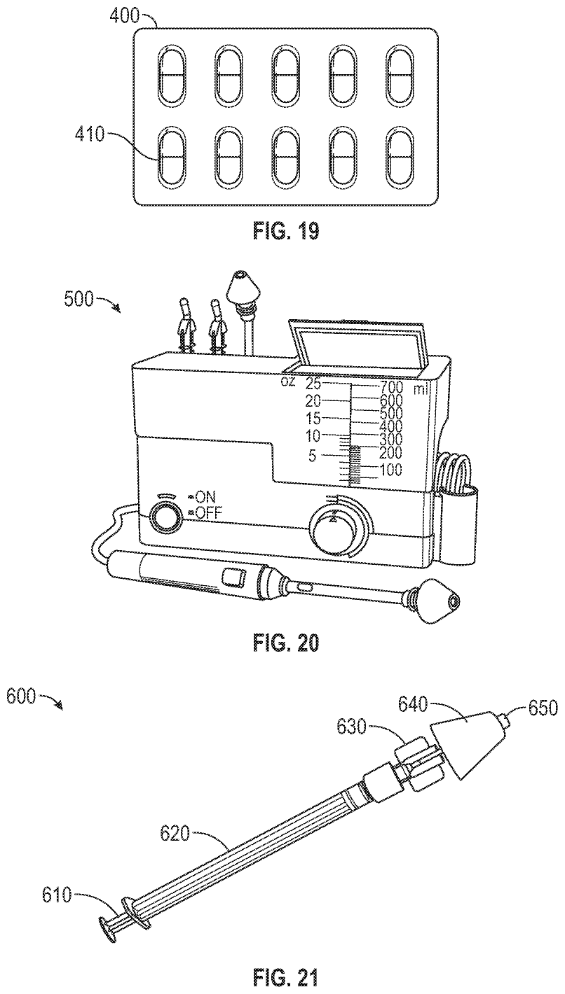

FIG. 18 illustrates a further medication delivery system 300 comprising a screw thread adapter 330 for attachment between a water bottle 310 and nosepiece 305. The nosepiece 305 includes threading 350 which allows the nose piece to attach to any standard water bottle via threading 340. In this embodiment, the screw thread adapter 330 includes interior 345 and exterior 340 threading in order to mate with opposite threading 335, 350 on the water bottle 310 and nosepiece 305, respectively. Similar to previous embodiments, the drug may be in the form of a tablet, powder, or gel formulation to be dissolved by dropping it into the water bottle 310.

FIG. 21 illustrates a MAD (Mucosal Atomization Device) 600 with a conical nose plug 640 attached. In operation, high pressure is applied to plunger 610 to ensure the dose is pressurized through syringe 620 and atomized into a fine mist of particles through the tip 650 of the nose plug 640. In some embodiments, the fine mist of particles may be 30-100 microns in size. A malleable stylet 630 allows for 180 degrees positioning of the nose plug 640, which forms a seal with the nostril, thus preventing expulsion of fluid.

According to some embodiments, any of the medication formulations described herein may include additional additives. Such additives may include various carbopol forms such as carbopol 934, carbomer homopolymer A, etc. Other additives may include but are not limited to hydroxypropyl methylcellulose (HPMC). HPMC is a semisynthetic, inert, viscoelastic polymer used as eye drops, as well as an excipient and controlled-delivery component in oral medicaments, found in a variety of commercial products. HPMC may help medication formulations adhere to the sinus and nasal mucosa longer to improve therapeutic outcomes.

Further additives may include Disodium EDTA for the disruption of biofilm by removing bivalent positive ions such as iron, calcium, zinc, and copper found in the bacterial biofilm matrix. Disodium EDTA is also used in some foods as a preservative or stabilizer to prevent catalytic oxidative and discoloration, which is catalyzed by metal ions. In addition, Disodium EDTA solutions are used to remove inorganic debris and lubricate the root canals in endodontics. Furthermore, Disodium EDTA solutions with the addition of a surfactant loosen up calcifications. At low concentrations Disodium EDTA has been shown to prevent biofilms by inhibiting the adhesion of bacteria. Furthermore, it has also been shown to reduce biofilm colonization and proliferation.

Additional additives may include a surfactant such as Polysorbate 80, Polysorbate 60, etc., an excipient used to stabilize aqueous formulations of medications. Surfactants such as polysorbate 80 and polysorbate 60 decrease surface tension improving the dissolution profile of the drug and the bioavailability of the final dosage.

In further embodiments, medication formulations may include the addition of a compound to preserve moisture such as propylene glycol, etc. Propylene glycol may also be used to assist in solubilizing various medications for topical use.

In other embodiments, medication formulations can include the addition of an emollient compound such as glycerin, etc. to assist in softening and moisturizing dry, crusty surfaces common with inflamed nasal and sinus tissues.

In additional embodiments, medication formulations may include the addition of certain muco-adhesive polymer compounds that bind to mucous epithelial cells such as carbopol formulations.

In one embodiment, the final irrigation volume per nostril is 5 cc to as high as 500 cc per nostril.

A further embodiment includes one or more polymers that adhere to the epithelial cell surface by binding to specific receptor sites such as certain lectins and thiolate polymers.

In various embodiments, other additives can include the addition of an anionic polymer such as sodium carboxymethyl cellulose, and various carbopols, etc.

In further embodiments, other additives include the addition of a cationic polymer such as chitosan, etc.

In some embodiments, medication formulations may include polymer ingredients such as: methyl cellulose, carboxymethyl cellulose, hydroxyethyl cellulose, hydroxypropyl cellulose, hydroxypropyl methyl cellulose, sodium carboxymethyl cellulose and various carbopol polymers.

In additional embodiments, other additives include the addition of various aloe compounds.

### Administration

In some embodiments of administration the medication will be in the dose form of a powder, granules, or pellets. These forms may or may not include effervescent properties. The administration may entail 1) mixing a packet of dry medication with a packet of dry saline and water, or 2) mixing a packet of dry medication which includes dry powdered saline and then mix with water, or 3) mixing a packet of dry medication with liquid saline. (FIG. 4) The patient may irrigate with any suitable irrigation device such as a syringe (FIG. 3), NeilMed® type rinse bottle 80 (FIG. 8), Hydropulse® 90 (FIG. 9), Sinupulse®, MAD (mucosal atomization device), Neti Pot 100 (FIG. 10), Navage® 110 (FIG. 11), or a standard purified water bottle with a screwed-on nosepiece attachment with or without an anti-siphon valve.

In some embodiments, water soluble hydrophyllic medicated gel is added to 1) a premixed saline solution or 2) a solution of saline prepared by mixing powdered salt and water. If the medicated gel formulation includes NaCl, then the gel is simply added to water. (FIG. 4) The patient may irrigate with any suitable irrigation device such as a syringe (FIG. 3), MAD (mucosal atomization device), NeilMed® type rinse bottle 80 (FIG. 8), Hydropulse® 90 (FIG. 9), Sinupulse®, Neti Pot 100 (FIG. 10), Navage® 110 (FIG. 11), or a standard purified water bottle with a screwed-on nosepiece attachment with or without an anti-siphon valve.

In some embodiments of administration, the medication will be in the dose form of a dissolvable effervescent tablet. This tablet may be in the form of a flat disc, round, oval, stick or dowel shaped tablet. The tablet may be small enough to fit through the opening of a NeilMed® type nasal irrigation bottle or a standard purified water bottle. An additional form may include medicated effervescent content contained in a capsule. The capsule is pulled apart and its content is added to water. This tablet and/or capsule dose form may or may not include NaCl. The administration may entail 1) Inserting a dissolvable effervescent medicated tablet into a mixture of previously prepared dry powdered saline and water, or 2) Inserting a dissolvable effervescent medicated tablet which includes NaCl into water, or 3) Inserting a dissolvable effervescent medicated tablet into liquid saline or 4) The patient may pull apart a capsule and empty its contents into water or liquid saline. The patient will then swirl the tablet or capsule contents and liquid until fully dissolved creating a uniform medicated mixture. (FIG. 4) The patient may then irrigate with any suitable irrigation device such as a syringe (FIG. 3), NeilMed® type rinse bottle 80 (FIG. 8), Sinupulse®, Hydropulse® 90 (FIG. 9), Neti Pot 100 (FIG. 10), Navage® 110 (FIG. 11), MAD (mucosal atomization device) or a standard purified water bottle with a screwed-on nosepiece attachment with or without an anti-siphon valve.

Multiple factors are associated with patient compliance with medicated nasal saline irrigation. Some of these factors include the preparing, mixing, and administering of the liquid medication. This invention provides for dosage standardization, which includes a pre-prepared and pre-measured dose of medication incorporated into a powder, gel, or tablet dose form. This invention also provides for an easy three-step process of preparation and administration: 1) Insert medication into liquid, 2) Swirl it around and 3) Irrigate nostrils. These dosage forms create a drug delivery system which is easier and more accurate than any previous system prescribed by physicians, formulated by compounding pharmacies or administered by patients. As a result, this process will improve patient compliance and improve patient outcomes. The drug standardization created by this invention will allow physicians to order a uniform product throughout the pharmacy industry, thus eliminating the need for special individual compounding of each order. In addition, the distribution of this product through retail pharmacies will also make it easier for a patient to obtain medicated nasal irrigation therapy. Widespread distribution will eventually reduce cost, making medicated irrigation therapy more accessible to patients.

Various embodiments have been described with reference to specific exemplary features thereof. It will, however, be evident that various modifications and changes may be made thereto without departing from the broader spirit and scope of the various embodiments as set forth in the appended claims. The specification and figures are, accordingly, to be regarded in an illustrative rather than a restrictive sense.

Although described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the present application, whether or not such embodiments are described and whether or not such features are presented as being a part of a described embodiment. Thus, the breadth and scope of the present application should not be limited by any of the above-described exemplary embodiments.

Terms and phrases used in the present application, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent.

Additionally, the various embodiments set forth herein are described in terms of exemplary diagrams and other illustrations. As will become apparent to one of ordinary skill in the art after reading this document, the illustrated embodiments and their various alternatives can be implemented without confinement to the illustrated examples. For example, diagrams and their accompanying description should not be construed as mandating a particular configuration.

What is claimed is:

1. A liquid topical sinus therapy for a patient, comprising:
a medicated formulation including at least one medication; and
a device for delivering the medicated formulation to the patient's nostrils, nasal passages, or sinuses.

2. The liquid topical sinus therapy of claim 1, wherein a liquid volume of the medicated formulation ranges from 5 ccs to 1000 ccs in a saline solution, which may be a hypotonic, isotonic, or hypertonic saline solution.

3. The liquid topical sinus therapy of claim 2, wherein the liquid volume includes drugs selected from the group consisting of: corticosteroids, antibiotics, antifungals, and antihistamines.

4. The liquid topical sinus therapy of claim 3, wherein a dose form of the drugs is manufactured to include effervescent properties.

5. The liquid topical sinus therapy of claim 3, wherein a dose form of the drugs is manufactured to include a medicated powder, granules, or pellets.

6. The liquid topical sinus therapy of claim 3, wherein a dose form of the drugs is manufactured to include a gel.

7. The liquid topical sinus therapy of claim 3, wherein a dose form of the drugs is manufactured to include a tablet or capsule.

8. The liquid topical sinus therapy of claim 3, wherein a dose form of the drugs is adapted to be dispensed in a standard purified water bottle.

9. The liquid topical sinus therapy of claim 1, wherein the device comprises a screw on nosepiece and tube that fits a standardized purified water bottle.

10. The liquid topical sinus therapy of claim 9, wherein the nosepiece and tube includes measure graduation marks.

11. The liquid topical sinus therapy of claim 1, wherein the medication comprises one or more corticosteroids selected from the group consisting of: mometasone furoate, mometasone furoate monohydrate, triamcinolone, beclomethasone, methylprednisolone, prednisolone, prednisone, betamethasone, ciclesonide, fluticasone furoate, fluticasone propionate, budesonide, cortisone, hydrocortisone, and dexamethasone.

12. The liquid topical sinus therapy of claim 1, wherein the medication comprises one or more antibiotics selected from the group consisting of: aminoglycosides, carbapenems, carboxylic acids, cephalosporins, fluoroquinolones, macrolides, monobactams, glycopeptides, tetracyclines, polypeptides, penicillins, sulfonamides and oxazolidinones.

13. The liquid topical sinus therapy of claim 1, wherein the medication comprises one or more antifungals selected from the group consisting of: polyene antifungals, azole antifungals, and echinocandins.

14. The liquid topical sinus therapy of claim 1, wherein the medication comprises one or more antihistamines selected from the group consisting of: Azelastine, Loratadine, Diphenhydramine, and Fexofenadine.

15. The liquid topical sinus therapy of claim 1, wherein the medication formulation comprises a coating adhesant selected from the group consisting of: carbopol, carbopol 934, homopolymer A, and hydroxypropyl methylcellulose (HPMC).

16. The liquid topical sinus therapy of claim 1, wherein the medication formulation comprises Disodium EDTA.

17. The liquid topical sinus therapy of claim 1, wherein the medication formulation comprises moisture emollient compounds selected from the group consisting of: propylene glycol and glycerin.

18. The liquid topical sinus therapy of claim 1, wherein the medication formulation comprises Chitosan.

19. The liquid topical sinus therapy of claim 1, wherein the medication formulation comprises polymer ingredients selected from the group consisting of: methyl cellulose, carboxymethyl cellulose, hydroxyethyl cellulose, sodium carboxymethyl cellulose and various carbopol polymers.

20. The liquid topical sinus therapy of claim 1, wherein the medication formulation comprises effervescent agents selected from the group consisting of: citric acid, sodium bicarbonate and tartaric acid.

21. The liquid topical sinus therapy of claim 1, wherein the medication formulation comprises an herbal additive selected from the group consisting of: menthol and eucalyptus.

22. The liquid topical sinus therapy of claim 1, wherein the medication formulation comprises an alternative medications selected from the group consisting of: manuka honey and methylglyoxal.
